(19) United States
(12) Patent Application Publication
(10) Pub. No.: US 2024/0245783 A1
(43) Pub. Date: Jul. 25, 2024

(54) METALLO-BETA-LACTAMASE INHIBITOR

(71) Applicant: NATIONAL UNIVERSITY CORPORATION KUMAMOTO UNIVERSITY, Kumamoto-shi, Kumamoto (JP)

(72) Inventors: Tomohiro SAWA, Kumamoto (JP); Yoshihiro YAMAGUTI, Kumamoto (JP); Yoshichika ARAKAWA, Aichi (JP)

(21) Appl. No.: 18/560,182

(22) PCT Filed: May 13, 2022
(86) PCT No.: PCT/JP2022/020285

§ 371 Date: Nov. 10, 2023

(30) Foreign Application Priority Data
May 13, 2021 (JP) 2021-081860

(51) Int. Cl. A61K 47/54; A61K 31/407; A61P 31/04

(52) U.S. Cl. CPC A61K 47/547; A61K 31/407; A61K 47/542; A61P 31/04

(57) ABSTRACT

A compound represented by formula (I), formula (II), or formula (IV), or a pharmaceutically acceptable salt thereof; and a pharmaceutical composition containing the compound or a pharmaceutically acceptable salt thereof, for use in inhibiting metallo-β-lactamases.

### TECHNICAL FIELD

The present invention relates to a compound having a β-lactam structure, a pharmaceutical composition for use in inhibiting metallo-β-lactamases, and a method for treating infectious diseases caused by β-lactam-resistant bacteria.

### BACKGROUND ART

In recent years, there have been many reports of infectious disease-causing bacteria that have acquired resistance to pi-lactam antibiotics, and thus the difficulty of treating such diseases is a problem. The most prominent resistance mechanism is the production of β-lactamases involved in degradation and inactivation of β-lactam antibiotics. β-lactamases are classified into class A, class B, class C and class D based on their primary amino acid sequences. β-lactamases belonging to class B are referred to as metallo-β-lactamases, and differ from serine-β-lactamases of the other classes having a serine residue in the active center (class A, class C and class D) in that they are metallo enzymes containing zinc in the active center.

Metallo-β-lactamases exhibit broad substrate specificity, and metallo-β-lactamase-producing bacteria are a threat because they become resistant to many clinically important β-lactam drugs. For example, metallo-β-lactamases hydrolyze carbapenem antibiotics, which are relatively stable against serine β-lactamases. In addition, metallo-β-lactamases have been confirmed in many bacterial species, and particularly Pseudomonas aeruginosa becoming multi-drug resistant through the production of metallo-β-lactamases is a problem. At present, those currently used as β-lactamase inhibitors are clavulanic acid, sulbactamn, tazobactam, etc., which are useful against serine β-lactamases, and inhibitors effective against metallo-β-lactamases have not been put into practical use.

Isolation and purification of metallo-β-lactamases have been reported (NON PATENT LITERATURE 5). In addition, as metallo-β-lactamase inhibitors, for example, succinic acid derivatives, maleic acid derivatives, phthalic acid derivatives and the like have been studied (PATENT LITERATURE 1 to 9). In addition, various compounds having metallo-β-lactamase inhibitory activity have been reported (PATENT LITERATURE 10 to 22 and NON PATENT LITERATURE 1 to 4).

### CITATION LIST

### Patent Literature

PTL 1: JP 2003-513890 A

PTL 2: JP 2003-527332 A

PTL 3: JP 2016-179964 A

PTL 4: JP 2008-115183 A

PTL 5: JP 2009-040743 A

PTL 6: JP 2013-032361 A

PTL 7: JP 2013-100289 A

PTL 8: WO 2007/034924 A1

PTL 9: WO 2008/016007 A1

PTL 10: WO 2013/015388 A1

PTL 11: JP 2016-538244 A

PTL 12: JP 2000-136133 A

PTL 13: JP 2000-143511 A

PTL 14: JP 2005-525399 A

PTL 15: JP 2000-504311 A

PTL 16: JP 2018-515481 A

PTL 17: JP H11-514981 A

PTL 18: JP 2016-520582 A

PTL 19: JP 2017-101027 A

PTL 20: JP 2017-132766 A

PTL 21: JP 2019-195315 A

PTL 22: JP 2000-336075 A

### Non Patent Literature

NPL 1: Yan. Y H. et al., Med Res Rev. 2020; 40:1558-1592.;

NPL 2: Palacios, A R., et al., Biomolecules 2020, 10, 854; doi:10.3390/biom10060854:

NPL 3: Jackson, A C. et al., Chem Med Chem 2021, 16, 654-661;

NPL 4: Wachino J. et al., mBio 2020, 11: e03144-19.

NPL 5: Osano E., et al., Antimicrobial Agents and Chemotherapy, January 1994, 71-78

### SUMMARY OF INVENTION

### Technical Problem

A new means has been required in treatment of infectious diseases caused by β-lactam-resistant bacteria, especially in treatment of infectious diseases caused by resistant bacteria that produce metallo-β-lactamases. An object of the present invention is to provide a metallo-β-lactamase inhibitor that can be used to suppress the inactivation of a β-lactam antibiotic by inhibiting metallo-β-lactamases.

### Solution to Problem

The present inventors have discovered a compound having metallo-β-lactamase inhibitory activity and completed the present invention. Description includes the disclosure of the following inventions.

[1] A compound represented by formula (I) or formula (II):

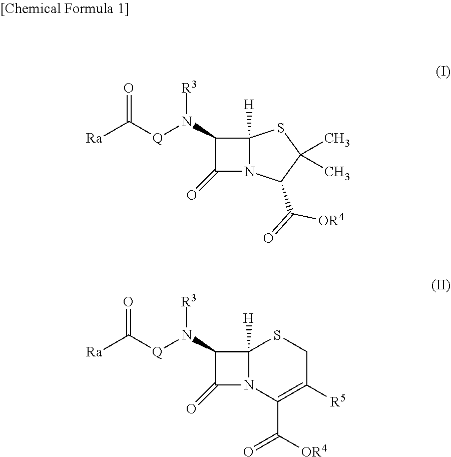

or a pharmaceutically acceptable salt thereof, wherein
Q is a direct bond or a group: —N(—R²)—CH(—R¹)—C(═O)—, wherein a nitrogen atom of the group is linked to a carbonyl group described in formula (I) or formula (II);
R¹ is phenyl optionally substituted with one or more substituents selected from X¹, 5- or 6-membered heteroaryl optionally substituted with one or more substituents selected from X¹, C₁₋₁₀ alkyl optionally substituted with one or more substituents selected from X², C₂₋₁₀ alkenyl optionally substituted with one or more substituents selected from X², C₂₋₁₀ alkynyl optionally substituted with one or more substituents selected from X², C₃₋₁₀ cycloalkyl optionally substituted with one or more substituents selected from X², or C₆₋₁₀ cycloalkanedienyl optionally substituted with one or more substituents selected from X³;
R² is a hydrogen atom or C₁₋₆ alkyl;
R³ is a hydrogen atom or C₁₋₆ alkyl;
R⁴ is a hydrogen atom, C₁₋₆ alkyl optionally substituted with one or more substituents selected from X⁴, or 5- or 6-membered non-aromatic heterocyclyloxy optionally substituted with one or more substituents selected from X⁵, wherein the heterocyclyl of the non-aromatic heterocyclyloxy is optionally fused with a benzene ring;
R⁵ is a hydrogen atom, a halogen atom, C₁₋₆ alkyl, C₁₋₆ alkoxy, C₂₋₆ alkenyl optionally substituted with R⁶, or methyl substituted with R⁷;
R⁶ is phenyl optionally substituted with one or more substituents selected from X¹, or 5- or 6-membered heteroaryl optionally substituted with one or more substituents selected from X¹;
R⁷ is 5- or 6-membered heteroarylsulfanyl optionally substituted with one or more substituents selected from X⁵, 5 or 6-membered heteroaryloxy optionally substituted with one or more substituents selected from X⁵, phenylsulfanyl optionally substituted with one or more substituents selected from X⁵, phenyloxy optionally substituted with one or more substituents selected from X⁵, (C₁₋₆ alkyl)carbonyloxy, carbamoyloxy, pyridinium-1-yl optionally fused with C₅₋₇ cycloalkyl and optionally substituted with X⁵, or 1-methylpyrrolidinium-1-yl optionally substituted with X⁵;
X¹ is amino, hydroxy, C₁₋₆ alkyl, C₁₋₆ alkoxy, a halogen atom, or phenyl optionally substituted with one or more halogen atoms or hydroxy;
X² is amino, hydroxy, C₁₋₆ alkoxy, a halogen atom, carboxy, or (C₁₋₆ alkoxy)carbonyl;
X³ is C₁₋₆ alkyl, or a halogen atom;
X⁴ is (C₁₋₆ alkyl)carbonyloxy or (C₁₋₆ alkoxy)carbonyloxy;
X⁵ is C₁₋₆ alkyl optionally substituted with R⁸, or carbamoyl;
R⁸ is hydroxy, sulfo, or carboxy; and
Ra is a polyamine group represented by any of the following formulae:

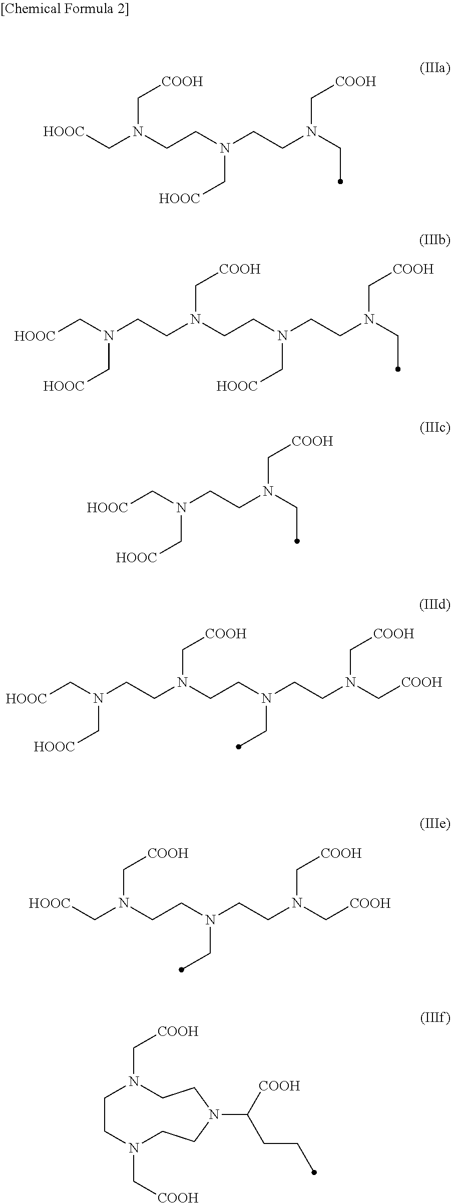

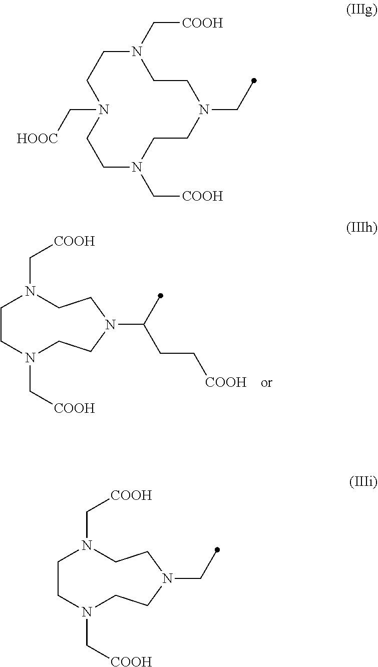

and ● indicates the bonding position.

[2] The compound or the pharmaceutically acceptable salt thereof according to [1], wherein R¹ is phenyl optionally substituted with one or more substituents selected from X¹, 5- or 6-membered heteroaryl optionally substituted with one or more substituents selected from X¹, or C₆₋₁₀ cycloalkanedienyl optionally substituted with one or more substituents selected from X³.

[3] The compound or the pharmaceutically acceptable salt thereof according to [1] or [2], wherein R⁴ is a hydrogen atom or C₁₋₆ alkyl substituted with one substituent selected from X⁴.

[4] The compound or the pharmaceutically acceptable salt thereof according to any of [1] to [3], wherein Ra is a polyamine group represented by formula IIIa, formula IIIh or formula IIIi.

[5] The compound or the pharmaceutically acceptable salt thereof according to any of [1] to [4], wherein R² is a hydrogen atom.

[6] The compound or the pharmaceutically acceptable salt thereof according to any of [1] to [5], wherein the compound is represented by formula II.

[7] The compound or the pharmaceutically acceptable salt thereof according to any of [1] to [6], wherein R⁵ is a hydrogen atom, a halogen atom, C₁₋₆ alkyl, C₁₋₆ alkoxy, or C₂₋₆ alkenyl.

[8] A pharmaceutical composition containing the compound or the pharmaceutically acceptable salt thereof according to any of [1] to [7].

[9] The pharmaceutical composition according to [8], for use in treating an infectious disease caused by a β-lactam antibiotic-resistant bacterium.

[10] The pharmaceutical composition according to [8] or [9], wherein the resistant bacterium is a metallo-β-lactamase-expressing bacterium.

[11] The pharmaceutical composition according to any of [8] to [10], for use in combination with a β-lactam antibiotic.

[12] A metallo-β-lactamase inhibitor, containing the compound or the pharmaceutically acceptable salt thereof according to any of [1] to [7].

[13] A method for treating an infectious disease caused by a β-lactam antibiotic-resistant bacterium, which involves administering the compound or the pharmaceutically acceptable salt thereof according to any of [1] to [7] to a subject in need of the treatment.

[14] The method according to [13], which further involves administering a therapeutically effective amount of a β-lactam antibiotic to the subject.

Description further includes the disclosure of the following inventions.

[A-1] A compound represented by formula (I), formula (II), or formula (IV):

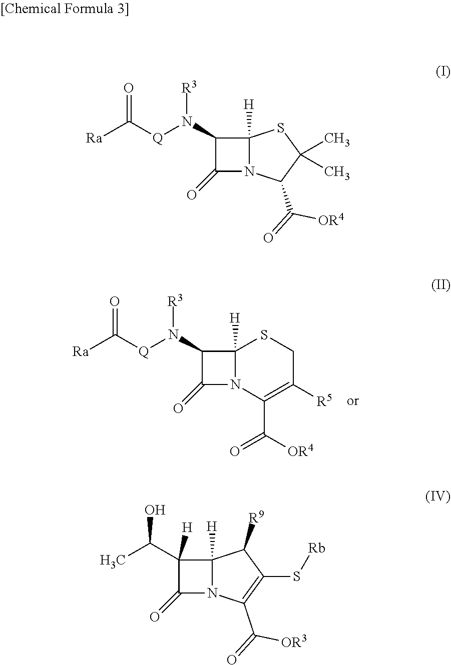

or a pharmaceutically acceptable salt thereof, wherein
Q is a direct bond or a group: —N(—R²)—CH(—R¹)—C(═O)—, wherein a nitrogen atom of the group is linked to a carbonyl group described in formula (I) or formula (II);
R¹ is phenyl optionally substituted with one or more substituents selected from X¹, 5- or 6-membered heteroaryl optionally substituted with one or more substituents selected from X¹, C₁₋₁₀ alkyl optionally substituted with one or more substituents selected from X², C₂₋₁₀ alkenyl optionally substituted with one or more substituents selected from X², C₂₋₁₀ alkynyl optionally substituted with one or more substituents selected from X², C₃₋₁₀ cycloalkyl optionally substituted with one or more substituents selected from X², or C₆₋₁₀ cycloalkanedienyl optionally substituted with one or more substituents selected from X³;
R² is a hydrogen atom or C₁₋₆ alkyl;
R³ is a hydrogen atom or C₁₋₆ alkyl;
R⁴ is a hydrogen atom, C₁₋₆ alkyl optionally substituted with one or more substituents selected from X⁴, or 5- or 6-membered non-aromatic heterocyclyloxy optionally substituted with one or more substituents selected from X⁵, wherein the heterocyclyl of the non-aromatic heterocyclyloxy is optionally fused with a benzene ring;
R⁵ is a hydrogen atom, a halogen atom, C₁₋₆ alkyl, C₁₋₆ alkoxy, C₂₋₆ alkenyl optionally substituted with R⁶, or methyl substituted with R⁷;
R⁶ is phenyl optionally substituted with one or more substituents selected from X¹ or 5- or 6-membered heteroaryl optionally substituted with one or more substituents selected from X¹;
R⁷ is 5- or 6-membered heteroarylsulfanyl optionally substituted with one or more substituents selected from X⁵, 5 or 6-membered heteroaryloxy optionally substituted with one or more substituents selected from X⁵, phenylsulfanyl optionally substituted with one or more substituents selected from X⁵, phenyloxy optionally substituted with one or more substituents selected from X⁵, (C₁₋₆ alkyl)carbonyloxy, carbamoyloxy, pyridinium-1-yl optionally fused with C₅₋₇ cycloalkyl and optionally substituted with X⁵, or 1-methylpyrrolidinium-1-yl optionally substituted with X⁵;
X¹ is amino, hydroxy, C₁₋₆ alkyl, C₁₋₆ alkoxy, a halogen atom, or phenyl optionally substituted with one or more halogen atoms or hydroxy;
X² is amino, hydroxy, C₁₋₆ alkoxy, a halogen atom, carboxy, or (C₁₋₆ alkoxy)carbonyl;
X³ is C₁₋₆ alkyl, or a halogen atom;
X⁴ is (C₁₋₆alkyl)carbonyloxy or (C₁₋₆ alkoxy)carbonyloxy;
X⁵ is C₁₋₆ alkyl optionally substituted with R⁸, or carbamoyl;
X⁶ is C₃₋₆ alkyl, a halogen atom, (C₁₋₆ alkoxy)carbonyl, or carboxy;
R⁸ is hydroxy, sulfo, or carboxy;
R⁹ is a hydrogen atom or methyl;
Ra is a polyamine group represented by any of the following formulae:

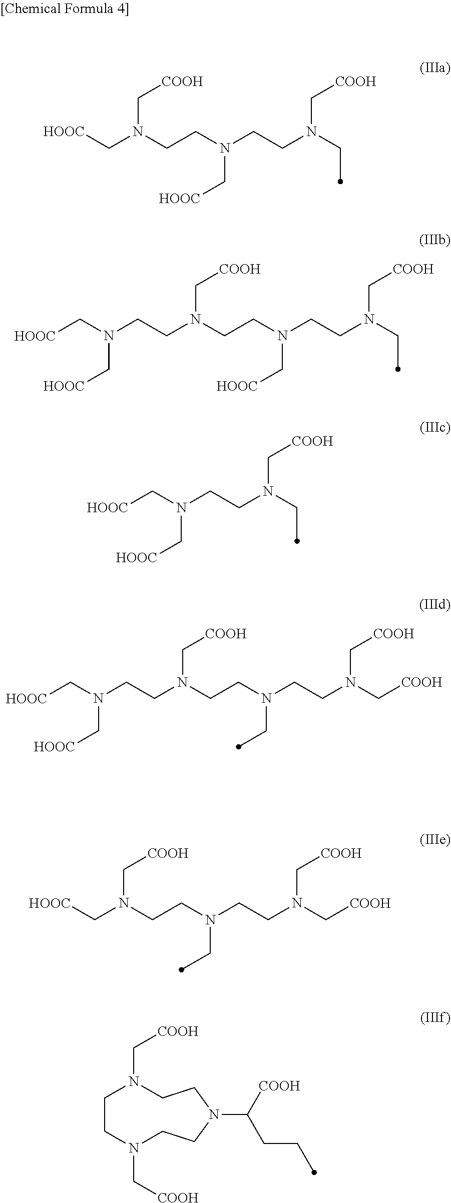

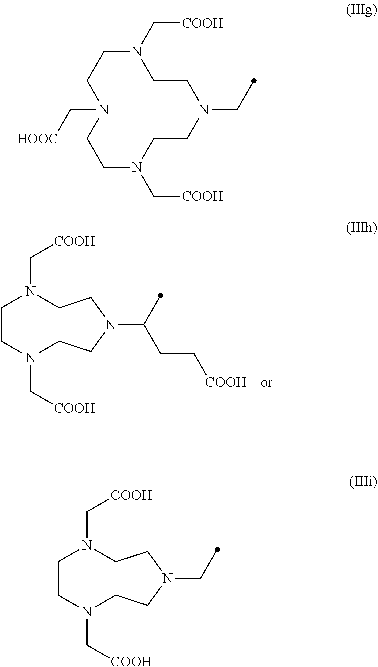

and ● indicates the bonding position; and
Rb is selected from groups represented by the following formulae:

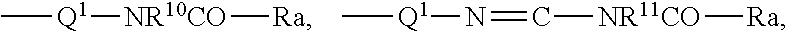

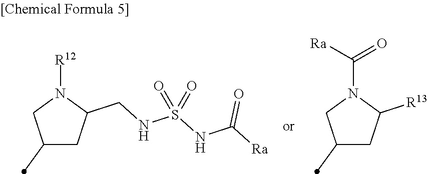

Q¹ is C₂₋₆ allylene;
R¹⁰ is a hydrogen atom, C₁₋₆ alkyl, or —CH═NH;
R¹¹ and R¹² are each independently a hydrogen atom or C₁₋₆ alkyl;
R¹³ is a hydrogen atom, C₁₋₆ alkyl, —CH₂NHSO₂NH₂, or —CONR¹⁴R¹⁵ wherein the alkyl is optionally substituted with one or more substituents selected from —NR¹⁴R¹⁵ and hydroxy;
R¹⁴ is a hydrogen atom, C₁₋₆ alkyl, or phenyl optionally substituted with one or more substituents selected from X⁶; and
R¹⁵ is a hydrogen atom or C₁₋₆ alkyl.

[A-2] The compound or the pharmaceutically acceptable salt thereof according to [A-1], wherein R¹ is phenyl optionally substituted with one or more substituents selected from X¹, 5 or 6-membered heteroaryl optionally substituted with one or more substituents selected from X¹, or C₆₋₁₀ cycloalkanedienyl optionally substituted with one or more substituents selected from X³.

[A-3] The compound or the pharmaceutically acceptable salt thereof according to [A-1] or [A-2], wherein R⁴ is a hydrogen atom, or C₁₋₆ alkyl substituted with one substituent selected from X⁴.

[A-4] The compound or the pharmaceutically acceptable salt thereof according to any of [A-1] to [A-3], wherein Ra is a polyamine group represented by formula IIIa, formula IIIh or formula IIIi.

[A-5] The compound or the pharmaceutically acceptable salt thereof according to any of [A-1] to [A-4], wherein R² is a hydrogen atom.

[A-6] The compound or the pharmaceutically acceptable salt thereof according to any of [A-1] to [A-5], wherein the compound is represented by formula II.

[A-7] The compound or the pharmaceutically acceptable salt thereof according to any of [A-1] to [A-6], wherein R⁵ is a hydrogen atom, a halogen atom, C₁₋₆ alkyl, C₁₋₆ alkoxy, or C₂₋₆ alkenyl.

[A-8] The compound or the pharmaceutically acceptable salt thereof according to any of [A-1] to [A-7], wherein Rb is selected from a group represented by the following formula:

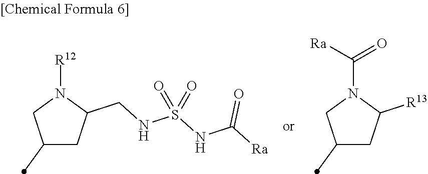

[A-9] The compound or the pharmaceutically acceptable salt thereof according to any of [A-1] to [A-8], wherein Rb is selected from a group represented by the following formula:

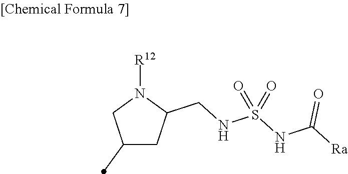

[A-10] A pharmaceutical composition containing the compound or the pharmaceutically acceptable salt thereof according to any of [A-1] to [A-9].

[A-11] The pharmaceutical composition according to [A-10], for use in treating an infectious disease caused by a β-lactam antibiotic-resistant bacterium.

[A-12] The pharmaceutical composition according to [A-10] or [A-11], wherein the resistant bacterium is a metallo-β-lactamase-expressing bacterium.

[A-13] The pharmaceutical composition according to any of [A-10] to [A-12], for use in combination with a β-lactam antibiotic.

[A-14] A metallo-β-lactamase inhibitor, containing the compound or the pharmaceutically acceptable salt thereof according to any of [A-1] to [A-9].

### Advantageous Effects of Invention

The present invention provides a metallo-β-lactamase inhibitor, and further provides a new means for treating infectious diseases caused by resistant bacteria that produce metallo-β-lactamases.

### BRIEF DESCRIPTION OF DRAWINGS

FIG. 1 depicts the results of purifying a reaction solution by reverse-phase HPLC in Example 1.

FIG. 2 depicts the results of confirming the molecular weight of a target compound by liquid chromatography-mass spectrometry (LC-MS).

FIG. 3 depicts the results of quantifying residual meropenem by tandem mass spectrometry in Test Example 1.

FIG. 4 is a graph showing the results of quantifying the peak areas of meropenem detected in FIG. 3.

FIG. 5 is a graph showing the results of quantifying residual meropenem by tandem mass spectrometry in Test Example 2.

FIG. 6 depicts the measurement results in Test Example 3. The results confirm the formation of a complex of DTPA-cefalexin and zinc.

FIG. 7 is a graph showing the results of quantifying residual meropenem by tandem mass spectrometry in Test Example 4.

FIG. 8 is a graph showing the susceptibility of IMP-1-expressing E. coli to meropenem when DTPA-cefalexin was added at 25 μM or EDTA was added at 25 μM.

FIG. 9 is a graph showing the susceptibility of IMP-1-expressing E. coli to meropenem when NOTA-cefalexin was added at 25 μM.

FIG. 10 depicts the MS spectrum of DTPA-cefachlor (compound 2).

FIG. 11 depicts the MS spectrum of DTPA-cephradine (compound 3).

FIG. 12 depicts the MS spectrum of DTPA-amoxicillin (compound 4).

FIG. 13 depicts the MS spectrum of NOTA-GA-cephradine (compound 5).

FIG. 14 depicts the MS spectrum of DTPA-ADCA (compound 6).

FIG. 15 depicts the MS spectrum of DTPA-cefalexin (compound 7).

FIG. 16 is a procedure for treating mice in Test Example 6.

FIG. 17 is a graph showing percent survival (%) of infected mice after drug administration in Test Example 6.

FIG. 18 is a graph showing the test results of confirming the growth inhibitory effect of compound 8 on a multidrug-resistant Pseudomonas aeruginosa strain in Test Example 8.

FIG. 19 is a graph showing the results of conducting a cytotoxicity test for the compounds of the present invention in Test Example 9.

FIG. 20 is a graph showing the test results of confirming the effect of DTPA-ADCA to enhance the antibacterial activity of meropenem against IMP-1-expressing E. coli (clinical isolate) in Test Example 10.

FIG. 21 depicts the MS spectrum of NODA-GA conjugated doripenem (compound 8).

### DESCRIPTION OF EMBODIMENTS

In one aspect, the present invention provides a compound represented by formula (I) formula (II) or formula (IV):

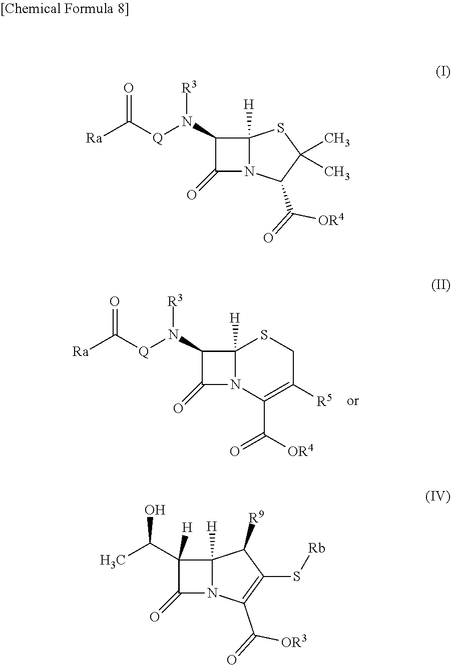

or a pharmaceutically acceptable salt thereof. Here, the compound represented by formula (I) includes compounds represented by the following formula (Ia) and formula (Ib). The compound represented by formula (II) includes a compound represented by the following formula (IIa) or formula (IIb).

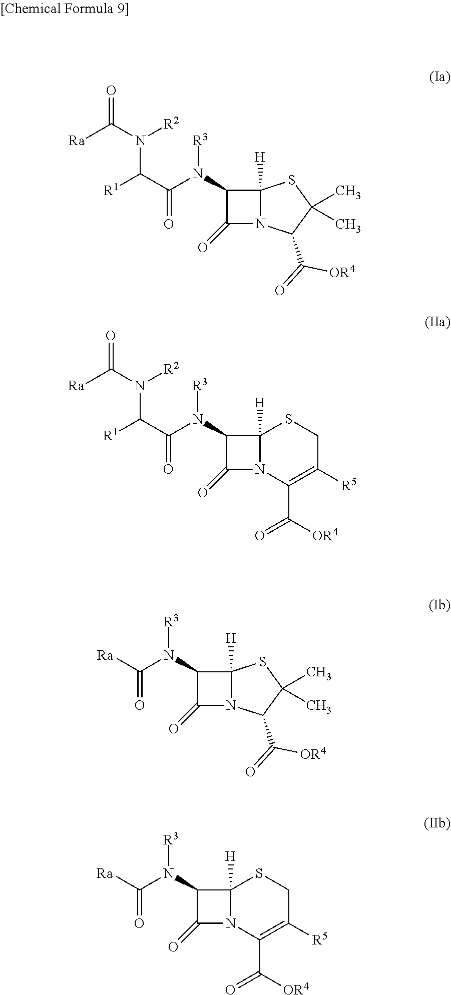

In one aspect, the present invention provides a compound represented by formula (I) or formula (II):

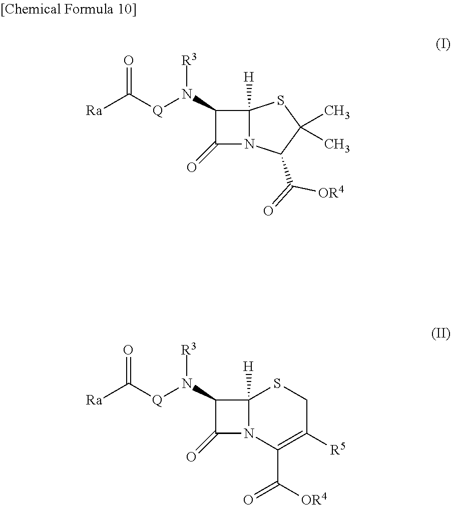

or a pharmaceutically acceptable salt thereof. In one embodiment, the present invention provides a compound represented by formula (Ia), formula (Ib), formula (IIa), or formula (IIb):

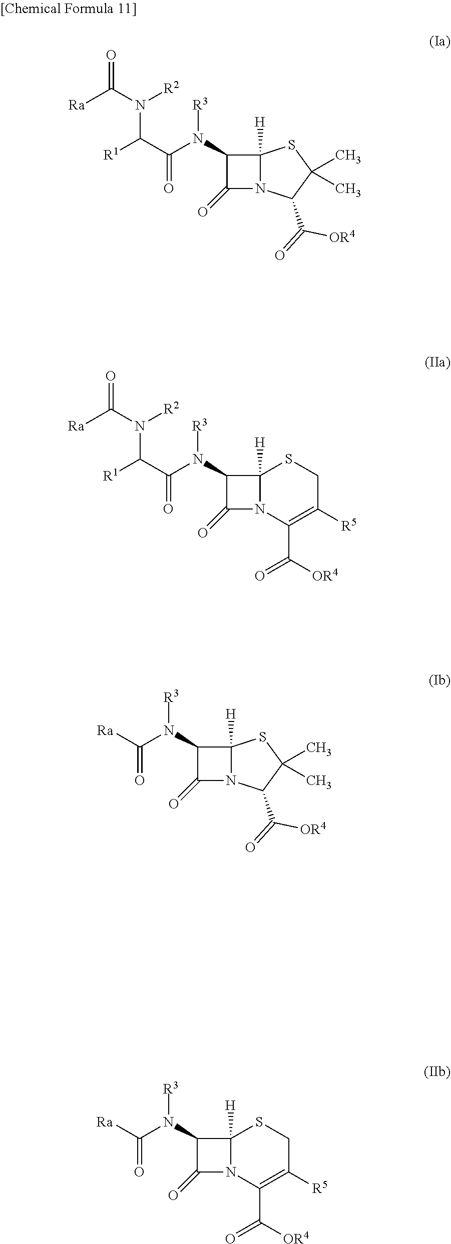

or a pharmaceutically acceptable salt thereof.

In one embodiment of the present invention, Rb is selected from groups represented by the following formulae:

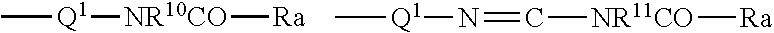

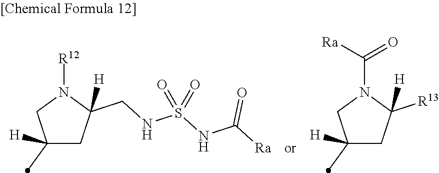

In one aspect of the present invention, examples of the compound represented by formula IV include the following compounds.

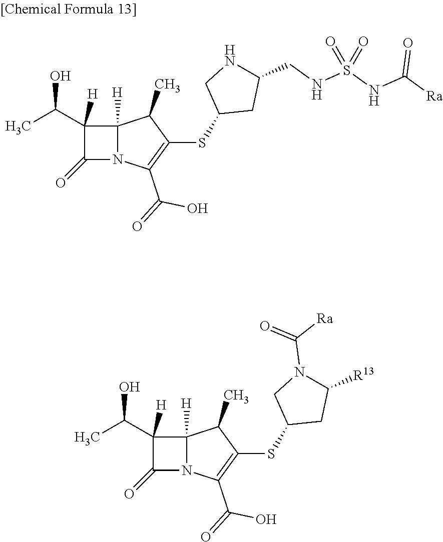

Here, examples of R¹³ include a hydrogen atom, (3-carboxyphenyl)aminocarbonyl, dimethylaminocarbonyl, aminosulfonylaminomethyl, and 3-aminomethyl-2-hydroxypropyl. More specific examples thereof include the following compounds:

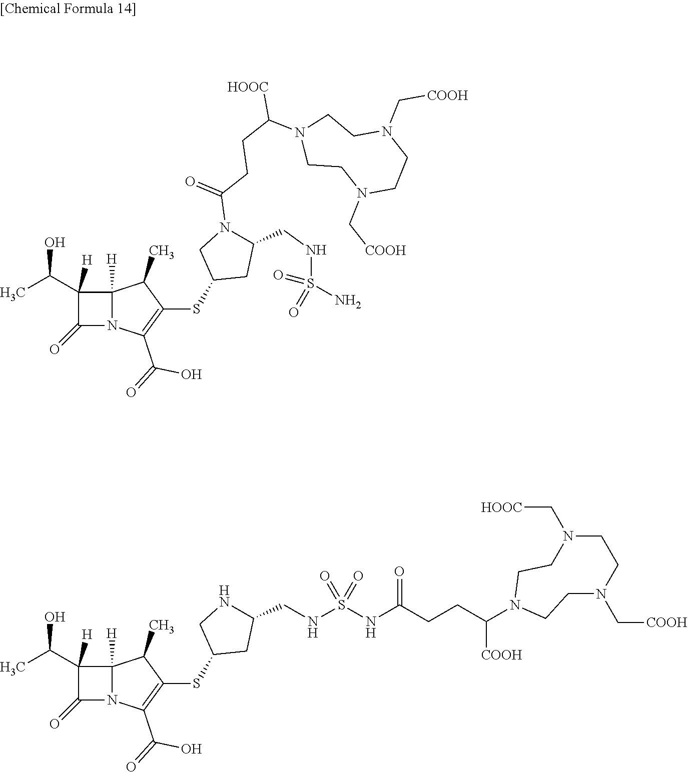 [SMILES: C[C@@H](O)[C@H]1C(=O)N2C(C(=O)O)=C(S[C@H]3C[C@@H](CNS(N)(=O)=O)N(C(=O)CCC(C(=O)O)N4CCN(CC(=O)O)CCN(CC(=O)O)CC4)C3)[C@H](C)[C@H]12 ; C[C@@H](O)[C@H]1C(=O)N2C(C(=O)O)=C(S[C@@H]3CN[C@H](CNS(=O)(=O)NC(=O)CCC(C(=O)O)N4CCN(CC(=O)O)CCN(CC(=O)O)CC4)C3)[C@H](C)[C@H]12]

As used herein, “C₁₋₁₀ alkyl” means a linear, branched, cyclic or partially cyclic alkyl group having 1 to 10 carbon atoms, and examples thereof include methyl, ethyl, n-propyl, i-propyl, n-butyl, s-butyl, i-butyl, t-butyl, n-pentyl, 3-methylbutyl, 2-methylbutyl, 1-methylbutyl, 1-ethylpropyl, n-hexyl, 4-methylpentyl, 3-methylpentyl, 2-methylpentyl, 1-methylpentyl, 3-ethylbutyl, 2-ethylbutyl, n-heptyl, n-octyl, n-nonyl, n-decanyl, cyclopropyl, cyclobutyl, cyclopentyl, cyclohexyl, and cyclopropylmethyl, and further include C₁₋₄ alkyl and C₁₋₃ alkyl.

As used herein, “C₁₋₆ alkyl” means a linear, branched, cyclic or partially cyclic alkyl group having 1 to 6 carbon atoms, and examples thereof include methyl, ethyl, n-propyl, i-propyl, n-butyl, s-butyl, i-butyl, t-butyl, n-pentyl, 3-methylbutyl, 2-methylbutyl, 1-methylbutyl, 1-ethylpropyl, n-hexyl, 4-methylpentyl, 3-methylpentyl, 2-methylpentyl, 1-methylpentyl, 3-ethylbutyl, and 2-ethylbutyl, cyclopropyl, cyclobutyl, cyclopentyl, cyclohexyl, and cyclopropylmethyl, and further include C₁₋₄ alkyl and C₁₋₃ alkyl.

As used herein, “C₁₋₆ alkoxy” means an alkyloxy group [—O—(C₁₋₆ alkyl)] having the alkyl group having 1 to 6 carbon atoms as already defined as an alkyl moiety, and examples thereof include methoxy, ethoxy, n-propoxy, i-propoxy, n-butoxy, s-butoxy, i-butoxy, t-butoxy, n-pentoxy, 3-methylbutoxy, 2-methylbutoxy, 1-methylbutoxy, 1-ethylpropoxy, n-hexyloxy, 4-methylpentoxy, 3-methylpentoxy, 2-methylpentoxy, 1-methylpentoxy, 3-ethylbutoxy, cyclopentyloxy, cyclohexyloxy, and cyclopropylmethyloxy, and further include C₁₋₄ alkoxy and C₁₋₃ alkoxy. Moreover, as used herein, examples of “C₁₋₄ alkoxy” include C₁₋₃ alkoxy, and the like.

As used herein, “C₂₋₁₀alkenyl” means a linear, branched, cyclic or partially cyclic alkenyl group having 2 to 10 carbon atoms, and has 1 or more, preferably 1 to 3, and further preferably one double bond. Examples of C₂₋₁₀ alkenyl include vinyl, 2-propenyl, 1-propenyl, 1-methylvinyl, 3-butenyl, 2-butenyl, and 1-butenyl.

As used herein, “C₂₋₆ alkenyl” means a linear, branched, cyclic or partially cyclic alkenyl group having 2 to 6 carbon atoms, and has 1 or more, preferably 1 to 3, and further preferably one double bond. Examples of C₂₋₁₀ alkenyl include vinyl, 2-propenyl, 1-propenyl, 1-methylvinyl, 3-butenyl, 2-butenyl, and 1-butenyl.

As used herein, “C₂₋₁₀alkynyl” means a linear, branched, cyclic or partially cyclic alkynyl group having 2 to 10 carbon atoms, and the alkynyl group has 1 or more, preferably 1 to 3, and further preferably one triple bond. Examples of C₂₋₆ alkynyl include ethynyl, 2-propynyl, 1-propynyl, 3-butynyl, 2-butynyl, and 1-butynyl.

As used herein, “C₆₋₁₀ cycloalkanedienyl” means a cyclic alkenyl group having 6 to 10 carbon atoms and two double bonds. Examples thereof include 1-cyclohexa-1,4-dienyl and 2-cyclohexa-1,4-dienyl.

As used herein, “C₃₋₁₀ cycloalkyl” means a cyclic alkyl group having 3 to 10 carbon atoms. Examples thereof include cyclopropyl, cyclobutyl, cyclopentyl, cyclohexyl, cycloheptyl, and cyclooctyl.

As used herein, “C₃₋₇ cycloalkyl” means a cyclic alkyl group having 3 to 7 carbon atoms. Examples thereof include cyclopropyl, cyclobutyl, cyclopentyl, cyclohexyl, and cycloheptyl.

As used herein, “(C₁₋₆ alkyl)carbonyl” means an alkylcarbonyl group having the C₁₋₆ alkyl group already defined as an alkyl moiety, and examples thereof include methylcarbonyl, ethylcarbonyl, and tert-butylcarbonyl, and further include (C₁₋₃ alkyl)carbonyl.

As used herein, “(C₁₋₆ alkoxy)carbonyl” means an alkoxycarbonyl group having the C₁₋₆ alkoxy group already defined as an alkoxy moiety, and examples thereof include methoxycarbonyl, ethoxycarbonyl, and tert-butoxycarbonyl, and further include (C₁₋₃ alkoxy)carbonyl.

As used herein, “(C₁₋₆ alkoxy)carbonyloxy” means an alkoxycarbonyl group having the (C₁₋₆ alkoxy)carbonyl group already defined as a (C₁₋₆ alkoxy)carbonyl moiety, and examples thereof include methoxycarbonyloxy, ethoxycarbonyloxy, and tert-butoxycarbonyloxy, and further include (C₁₋₃ alkoxy)carbonyloxy.

As used herein, “5- or 6-membered ring heteroaryl” is not particularly limited, as long as it is a 5-membered or 6-membered ring aromatic heterocyclic group containing 1 or more, for example 1 to 4, or 1 to 3 heteroatoms selected from oxygen, nitrogen, and sulfur atoms. The heteroaryl group may be the one wherein the ring-constituting carbon is a carbonyl group. Examples thereof include pyridyl, pyrimidyl, pyridazinyl, pyrazyl, furanyl (furyl), thiophenyl (thienyl), oxazolyl, isoxazolyl, oxadiazolyl, thiazolyl, isothiazolyl, thiadiazolyl, pyrrolyl, imidazolyl, pyrazolyl, triazolyl, tetrazolyl, and 5-oxo-2,5-dihydro-1,2,4-triazine.

As used herein, “5- or 6-membered ring heteroaryloxy” is a 5- or 6-membered ring heteroaryloxy having the 5- or 6-membered ring heteroaryl already defined as a 5- or 6-membered ring heteroaryl moiety. Examples thereof include pyridyloxy, pyrimidyloxy, pyridazinioxyl, pyrazyloxy, furanyloxy (furyloxy), thiophenyloxy (thienyloxy), oxazolyloxy, isoxazolyloxy, oxadiazolyloxy, thiazolyloxy, isothiazolyloxy, thiadiazolyloxy, pyrrolyloxy, imidazolyloxy, pyrazolyloxy, triazolyloxy, tetrazolyloxy, and 5-oxo-2,5-dihydro-1,2,4-triazinoxy.

As used herein, “5- or 6-membered ring heteroarylsulfanyl” is a 5- or 6-membered ring heteroarylsulfanyl [(5- or 6-membered ring heteroaryl)-S-] having the 5- or 6-membered ring heteroaryl already defined as a 5- or 6-membered ring heteroaryl moiety. Examples thereof include pyridylsulfanyl, pyrimidylsulfanyl, pyridazinylsulfanyl, pyrazylsulfanyl, furanylsulfanyl (furylsulfanyl), thiophenylsulfanyl (thienylsulfanyl), oxazolylsulfanyl, isoxazolvsulfanyl, oxadiazolylsulfanyl, thiazolylsulfanyl, isothiazolylsulfanyl, thiadiazolylsulfanyl, pyrrolylsulfanyl, imidazolylsulfanyl, pyrazolylsulfanyl, triazolylsulfanyl, tetrazolylsulfanyl, and 5-oxo-2,5-dihydro-1,2,4-triazinesulfanyl.

As used herein, “5- or 6-membered non-aromatic heterocyclyloxy” means non-aromatic heterocyclyloxy containing a non-aromatic heterocyclic group containing 1 or more, for example 1 to 4, or 1 to 3 heteroatoms selected from nitrogen, oxygen and sulfur atoms as 5- or 6-membered non-aromatic heterocyclyl. Examples thereof include tetrahydrofuranyloxy, dihydrofuranyloxy, pyrrolidinyloxy, piperidinyloxy, piperazinyloxy, and morpholinyloxy.

Examples of halogen atoms include fluorine, chlorine, bromine, and iodine atoms.

As used herein, “sulfo” represents the group: —SO₂OH.

As used herein, when R or the like is optionally substituted with one or more substituents selected from any of X¹ to X⁵, the number of substituents ranges from 1 to 4, or 1 to 3, or is 1 or 2, or 1. Moreover, when a plurality of substituents are present, the substituents may be the same or different.

As used herein, “pharmaceutically acceptable salt” is not particularly limited as long as it is a salt that can be used as a pharmaceutical. Examples of a salt formed by the compound of the present invention with a base include a salt formed with an inorganic base such as sodium, potassium, magnesium, calcium and aluminum; and a salt formed with an organic base such as methylamine, ethylamine and ethanolamine. The salt may be an acid addition salt, and specific examples of such a salt include acid addition salts formed with mineral acids such as hydrochloric acid, hydrobromic acid, hydroiodic acid, sulfuric acid, nitric acid, and phosphoric acid; and organic acids such as formic acid, acetic acid, propionic acid, oxalic acid, malonic acid, succinic acid, fumaric acid, maleic acid, lactic acid, malic acid, tartaric acid, citric acid, methanesulfonic acid and ethanesulfonic acid.

In one embodiment of the present invention, the compound represented by formula (I), formula (II) or formula (IV) may be present as a pharmaceutically acceptable salt, and some or all of groups capable of forming salts, such as a carboxy group contained in the compound may form salts. Also, when a cation such as a pyridinium group is present in the compound, an intramolecular carboxy group may be a counter anion, or another counter anion may also be present.

Atoms contained in the compound represented by formula (I), formula (II) or formula (IV) (for example, a hydrogen atom, a carbon atom, an oxygen atom, a nitrogen atom, and a sulfur atom) may each be an isotope atom other than the most abundant naturally-occurring isotope, or the isotope atom may be a radioactive isotope atom. Thus, according to one aspect of the present invention, a compound represented by formula (I), formula (II) or formula (IV), or a salt thereof, as previously defined herein, which is labeled with the isotope atom is provided. Here, the labeling with an isotope atom may be, for example, labeling with a radioactive isotope (³H, ¹⁴C, ³²P, etc.). Labeling with ³H is preferred in view of the ease of preparing the compound.

In one embodiment of the present invention, the compound represented by formula (I), formula (II) or formula (IV), an enantiomer thereof, a diastereomer thereof, or a pharmaceutically acceptable salt thereof is administered as a prodrug and converted to an active compound in vivo.

In one aspect of the present invention, the pharmaceutical composition can be formulated in various dosage forms, such as tablets, capsules, powders, granules, pills, liquid agents, emulsions, suspensions, solutions, spirits, syrups, extracts, or elixirs for oral administration. The pharmaceutical composition of the present invention can be formulated in the form of parenteral agents including injections such as subcutaneous injections, intravenous injections, intramuscular injections and intraperitoneal injections; adhesive patches, ointments or lotions for transdermal administration; sublingual formulations, and buccal patches for buccal administration; and aerosol formulations for nasal administration, but the examples thereof are not limited thereto. These formulations can be produced by known methods commonly used in formulation processes.

The pharmaceutical composition can contain various commonly used ingredients, for example, one or more types of pharmaceutically acceptable excipients, disintegrants, diluents, lubricants, aromatic agents, coloring agents, sweetening agents, corrigents, suspending agents, wetting agents, emulsifying agents, dispersing agents, adjuvants, preservatives, buffering agents, binders, stabilizers, coating agents and the like. The pharmaceutical composition of the present invention may also be in a sustained or sustained release dosage form.

In one aspect of the present invention, the dosage of the pharmaceutical composition can be appropriately selected depending on the route of administration, a patient's body type, age, and physical conditions, the degree of the disease, elapsed time after the onset, and the like. The pharmaceutical composition of the present invention can contain a therapeutically effective amount and/or a prophylactically effective amount of the compound represented by formula (I), formula (II) or formula (IV) above. In the present invention, the compound represented by formula (I), formula (II) or formula (IV) above can generally be used in doses of 1-1000 mg/day/adult or 0.01-20 mg/day/kg body weight. The pharmaceutical composition may be administered in a single dose or multiple doses.

In the composition for oral administration containing the compound of the present invention, the content of the compound ranges from, for example, 0.001 mg to 1000 mg, specifically 0.01 mg to 500 mg, and particularly specifically 0.005 mg to 100 mg per unit dosage form.

The pharmaceutical composition of the present invention may contain conventionally known ingredients, such as coloring agents, preservatives, fragrances, flavoring agents, coating agents, antioxidants, vitamins, amino acids, peptides, proteins, and minerals (iron, zinc, magnesium, iodine, etc.), as necessary. In one embodiment of the present invention, the pharmaceutical composition may be prepared in a form suitable for oral administration, such as various solid formulations including granules (including dry syrup), capsules (soft capsules, hard capsules), tablets (including chewable tablets, etc.), powders (powder formulations) and pills, or liquid formulations including liquid agents for internal use (including liquid agents, suspensions, and syrups).

Examples of additives for formulation include excipients, lubricants, binders, disintegrants, fluidizing agents, dispersing agents, wetting agents, preservatives, thickeners, pH adjusters, coloring agents, corrigents and deodorants, surfactants, and solubilizing agents. Moreover, upon preparation in the form of a liquid agent, thickening agents, such as pectin, xanthan gum, and guar gum, can be blended. Further, a coating agent can be used to prepare a coated tablet or a paste-like glue. Furthermore, upon preparation in other forms, conventional methods may be followed.

According to one aspect of the present invention, the compound represented by formula (I), formula (II) or formula (TV), or a pharmaceutically acceptable salt thereof, is used as a metallo-β-lactamase inhibitor, wherein the metallo-β-lactamase inhibitor is administered in combination with a β-lactam antibiotic. In one embodiment of the present invention, the metallo-β-lactamase inhibitor is administered simultaneously, separately, or sequentially with the β-lactam antibiotic.

Examples of the β-lactam antibiotic include carbapenems, penicillins, cephems, or prodrugs thereof.

Examples of carbapenems include imipenem, meropenem, biapenem, doripenem, ertapenem, tebipenem pivoxil, and tomopenem (CS-023). More specific examples of carbapenems include imipenem, meropenem, biapenem and doripenem.

Examples of penicillins include benzylpenicillin, phenoxymethylpenicillin, carbenicillin, azidocillin, propicillin, ampicillin, amoxicillin, epicillin, ticarcillin, cyclacillin, pyrbenicillin, azlocillin, mezlocillin, sulbenicillin, piperacillin and other known penicillins, and prodrugs thereof.

Examples of cephems include cefatrizine, cephalorizine, cephalothin, cefazolin, cefalexin, cefacetril, cefapirin, cefamandole nafate, cephradine, 4-hydroxycefalexin, cefoperazone, latamoxef, cefminox, flomoxef, cefsulodin, ceftazidime, cefuroxime, cefditoren, cefmetazole, cefotaxime, ceftriaxone, cefepime, cefpirome, cefozopran, and prodrugs thereof.

According to one embodiment of the present invention, other types of antibiotics may be used in addition to β-lactam antibiotics.

According to one embodiment of the present invention, other β-lactamase inhibitors may be used in combination, in addition to the metallo-β-lactamase inhibitor. Preferred examples thereof include serine-β-lactamase inhibitors such as clavulanic acid, sulbactam or tazobactam.

In one aspect of the present invention, metallo-β-lactamase inhibitors are used for treating infectious diseases caused by metallo-β-lactamase-producing strains. Examples of metallo-β-lactamase-producing strains include Bacillus cereus, Bacteroides fragilis, Escherichia coli, Aeromonas hydrophila, Klebsiella pneumoniae, Pseudomonas aeruginosa, Serratia marcescens, Stenotrophomonas maltophilia, Shigella flexneri, Alcaligenes xylosoxidans, Legionella gormanii, Chryseobacterium meningosepticum, Chryseobacterium indologenes, Acinetobacter baumannii, Citrobacter freundii, and Enterobacter cloacae.

The dosages of the compound represented by formula (I), formula (II) or formula (IV) or a pharmaceutically acceptable salt thereof and the antibiotic can vary within wide limits, but, for example, the weight ratio of the dosages is generally about 1:0.5 to 1:20, and preferably 1:1 to 1:8.

The metallo-β-lactamase inhibitor and the β-lactam antibiotic can be administered separately, or can be administered in the form of a single composition containing both active ingredients. In any of the embodiments, the compound represented by formula (I), formula (II) or formula (IV), or a pharmaceutically acceptable salt thereof, is preferably formulated into a pharmaceutical composition through combination with an antibiotic and a pharmaceutically acceptable carrier (i.e., pharmaceutical additive).

The compound represented by formula (I), formula (II) or formula (IV) can be synthesized by reacting a β-lactam compound having an amino group with a carboxylic acid corresponding to polyamine groups represented by formulae IIIa to IIIi. In one embodiment, the reaction can be carried out by reacting a carboxylic acid anhydride with a β-lactam compound. When the acid anhydride is a divalent acid anhydride, the dimer produced as a by-product can be separated to obtain the target compound. In another embodiment, the target product can be obtained by reacting the carboxylic acid with a β-lactam compound in the presence of a condensing agent.

The present invention is as illustrated in Examples below, but Examples are not intended to limit the present invention.

### [Example 1] Synthesis of DTPA-Conjugated Cefalexin

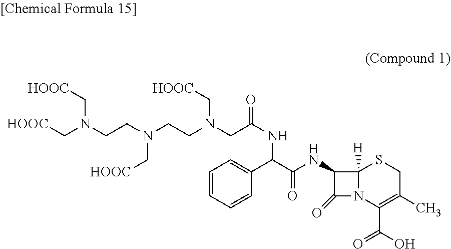 [SMILES: CC1=C(C(=O)O)N2C(=O)[C@@H](NC(=O)C(NC(=O)CN(CCN(CCN(CC(=O)O)CC(=O)O)CC(=O)O)CC(=O)O)c3ccccc3)[C@H]2SC1]

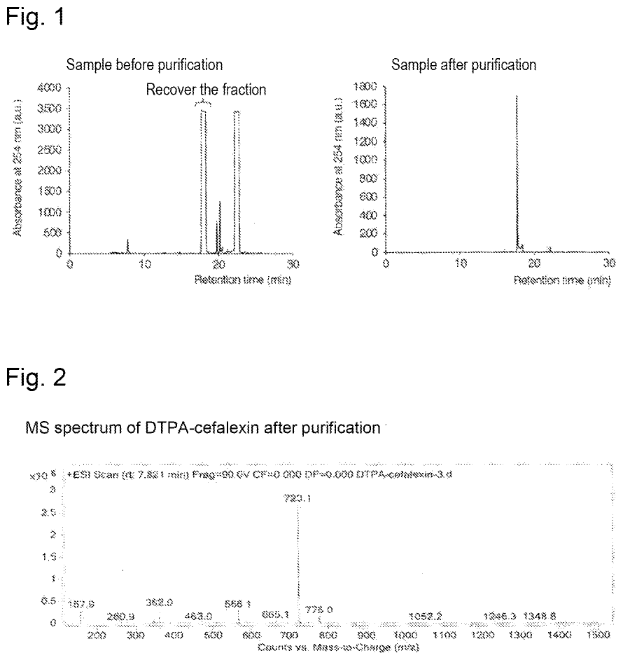

Cefalexin (Wako Pure Chemical Industries, Ltd.) was dissolved in a 400 mM disodium hydrogen phosphate aqueous solution in such a manner that the concentration was 20 mM. Powdered anhydrous DTPA (Dojindo Laboratories) (formula below) was added to the solution in such a manner that the final concentration was 20 mM, thereby performing 30 minutes of reaction at 37° C. The reaction solution was purified by reverse-phase HPLC under the following conditions to obtain compound 1 (FIG. 1, yield: 47%).

Reverse phase column: YMC-Triat C18 Plus column (4.6×250 mm); column temperature: 35° C.; mobile phase (2 liquids): mobile phase A (0.1% formic acid aqueous solution), mobile phase B (acetonitrile); gradient: increase from 0.2% B to 40% B for 22 minutes, keep it at 40% B for 1 minute, and then return it to 0.2% B over 1 minute. Flow rate: 0.8 ml/min; detection: 254 nm; sample injection volume: 1 ml.

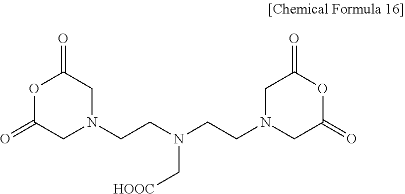 [SMILES: O=C(O)CN(CCN1CC(=O)OC(=O)C1)CCN1CC(=O)OC(=O)C1]

The eluted fraction containing the target compound (compound 1, molecular weight: 722.72) was collected (FIG. 1) and lyophilized. The molecular weight of the compound contained in the fraction was confirmed by liquid chromatography-mass spectrometry (LC-MS) (FIG. 2).

### [Example 2] Synthesis of DTPA-Conjugated Cefaclor

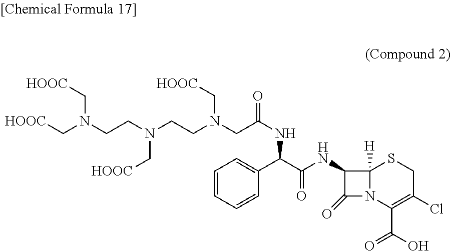 [SMILES: O=C(O)CN(CCN(CC(=O)O)CC(=O)O)CCN(CC(=O)O)CC(=O)N[C@@H](C(=O)N[C@@H]1C(=O)N2C(C(=O)O)=C(Cl)CS[C@H]12)c1ccccc1]

Compound 2 was prepared by the same method as in Example 1 using cefaclor (Sigma-Aldrich) (yield: 43%). FIG. 10 depicts the results of confirming the molecular weight of the target compound by liquid chromatography-mass spectrometry (LC-MS).

### [Example 3] Synthesis of DTPA-Conjugated Cephradine

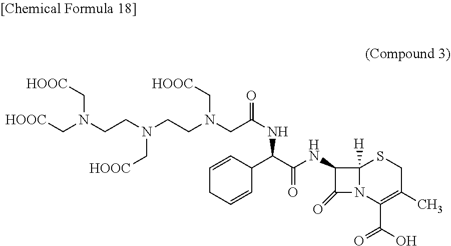 [SMILES: CC1=C(C(=O)O)N2C(=O)[C@@H](NC(=O)[C@H](NC(=O)CN(CCN(CCN(CC(=O)O)CC(=O)O)CC(=O)O)CC(=O)O)C3C=CCC=C3)[C@H]2SC1]

Compound 3 was prepared by the same method as in Example 1 using cephradine (Sigma-Aldrich) (yield: 27%). FIG. 11 depicts the results of confirming the molecular weight of the target compound by liquid chromatography-mass spectrometry (LC-MS).

### [Example 4] Synthesis of DTPA-Conjugated Amoxicillin

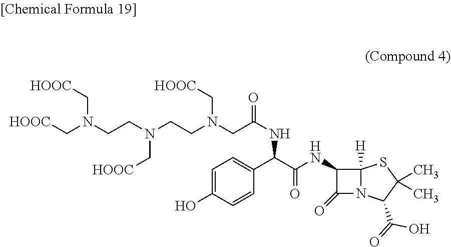 [SMILES: CC1(C)S[C@@H]2[C@H](NC(=O)[C@H](NC(=O)CN(CCN(CCN(CC(=O)O)CC(=O)O)CC(=O)O)CC(=O)O)c3ccc(O)cc3)C(=O)N2[C@H]1C(=O)O]

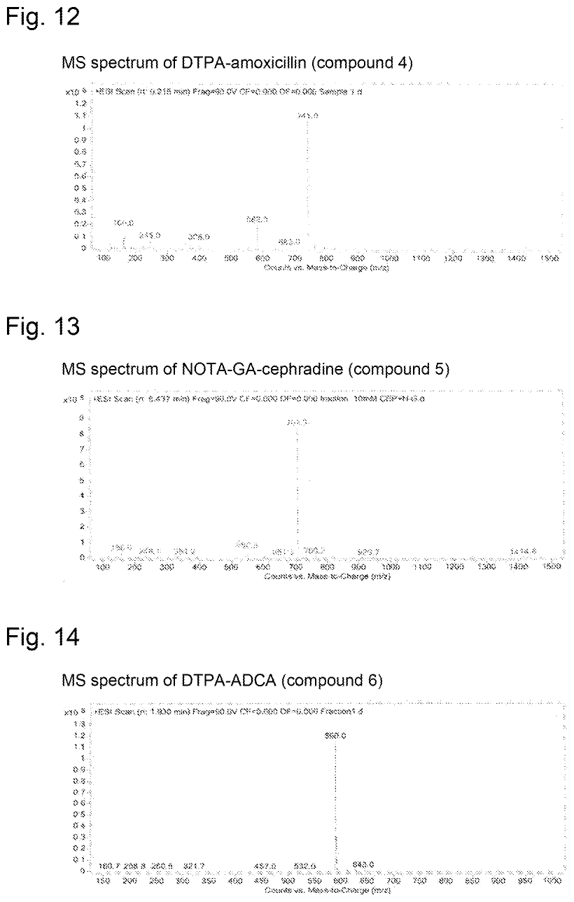

Compound 4 was prepared by the same method as in Example 1 using amoxicillin (Fujifilm) (yield: 41%). FIG. 12 depicts the results of confirming the molecular weight of the target compound by liquid chromatography-mass spectrometry (LC-MS).

### [Example 5] Synthesis of NOTA-GA-Conjugated Cephradine

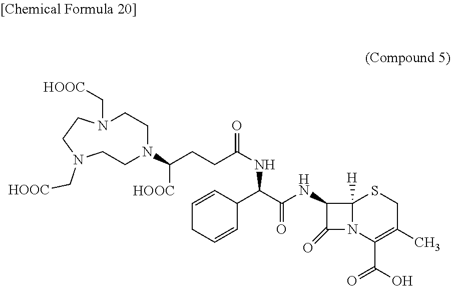 [SMILES: CC1=C(C(=O)O)N2C(=O)[C@@H](NC(=O)[C@H](NC(=O)CC[C@@H](C(=O)O)N3CCN(CC(=O)O)CCN(CC(=O)O)CC3)C3C=CCC=C3)[C@H]2SC1]

Cephradine (Sigma-Aldrich) was dissolved in a 400 mM disodium hydrogen phosphate aqueous solution in such a manner that the concentration was 20 mM. Powdered NOTA-GA-NHS (2,2′-(7-(1-carboxy-4-((2,5-dioxopyrrolidin-1-yl)oxy)-4-oxobutyl)-1,4,7-triazonane-1,4-diyl)diacetic acid; CheMatech, France) was added to the solution in such a manner that the final concentration was 30 mM, thereby performing 30 minutes of reaction at 37° C. The reaction solution was purified by reverse-phase HPLC under the following conditions to obtain compound 5 (yield: 85%).

Reverse phase column: YMC-Triat C18 Plus column (4.6×250 mm); column temperature: 35° C.; mobile phase (2 liquids): mobile phase A (0.1% formic acid aqueous solution), mobile phase B (acetonitrile); gradient: increase from 0.2% B to 40% B for 22 minutes, keep it at 40% B for 1 minute, and then return it to 0.2% B over 1 minute. Flow rate: 0.8 ml/min; detection: 254 nm; sample injection volume: 1 ml.

FIG. 13 depicts the results of confirming the molecular weight of the target compound by liquid chromatography-mass spectrometry (LC-MS).

### [Example 6] Synthesis of DTPA-Conjugated ADCA

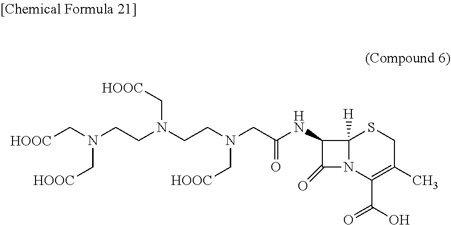 [SMILES: CC1=C(C(=O)O)N2C(=O)[C@@H](NC(=O)CN(CCN(CCN(CC(=O)O)CC(=O)O)CC(=O)O)CC(=O)O)[C@H]2SC1]

Compound 6 was prepared by the same method as in Example 1 using 7-aminodesacetoxycephalosporanic acid (7-ADCA, Tokyo Chemical Industry, Ltd.) (yield: 58%). FIG. 14 depicts the results of confirming the molecular weight of the target compound by liquid chromatography-mass spectrometry (LC-MS).

### [Example 7] Synthesis of NOTA-Conjugated Cefalexin

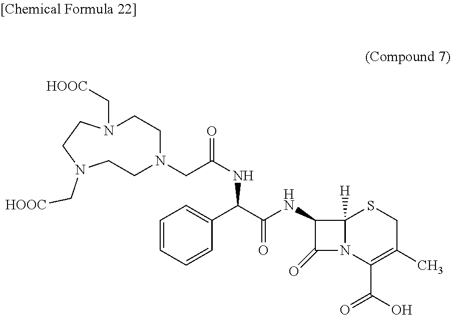 [SMILES: CC1=C(C(=O)O)N2C(=O)[C@@H](NC(=O)[C@H](NC(=O)CN3CCN(CC(=O)O)CCN(CC(=O)O)CC3)c3ccccc3)[C@H]2SC1]

Cefalexin (Wako Pure Chemical Industries, Ltd.) was dissolved in a 400 mM disodium hydrogen phosphate aqueous solution in such a manner that the concentration was 30 mM. Powdered NOTA-NHS (2,2′-(7-(2-((2,5-dioxopyrrolidin-1-yl)oxy)-2-oxoethyl)-1,4,7-triazonane-1,4-diyl)diacetic acid (CheMatech)) was added to the solution in such a manner that the final concentration was 40 mM, thereby performing 30 minutes of reaction at 37° C. The reaction solution was purified by reverse-phase HPLC under the following conditions to obtain compound 7 (yield: 82%).

Reverse phase column: YMC-Triat C18 Plus column (4.6×250 mm); column temperature: 35° C.; mobile phase (2 liquids): mobile phase A (0.1% formic acid aqueous solution), mobile phase B (acetonitrile); gradient: increase from 0.2% B to 40% B for 22 minutes, keep it at 40% B for 1 minute, and then return it to 0.2% B over 1 minute. Flow rate: 0.8 mil/min; detection: 254 nm; sample injection volume: 1 ml.

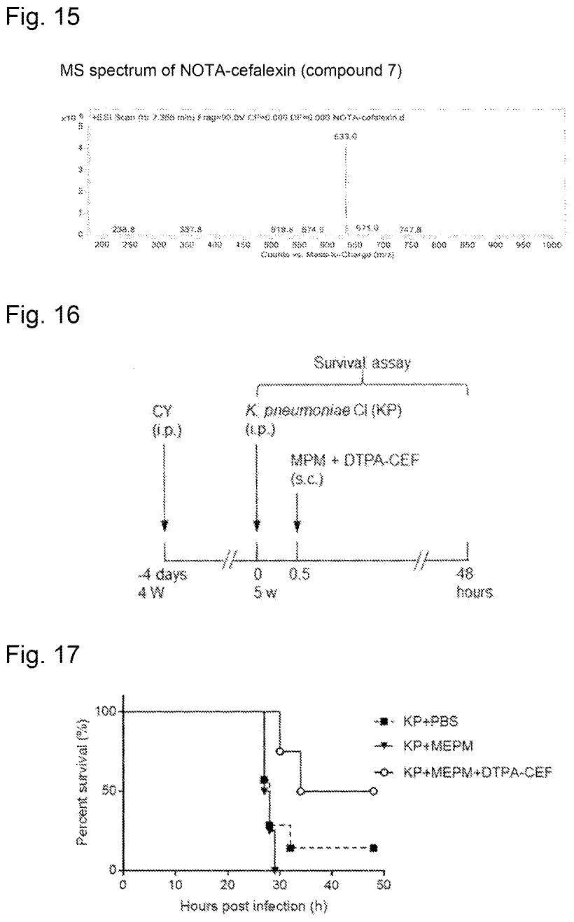

FIG. 15 depicts the results of confirming the molecular weight of the target compound by liquid chromatography-mass spectrometry (LC-MS).

### [Example 8] Synthesis of NODA-GA Conjugated Doripenem

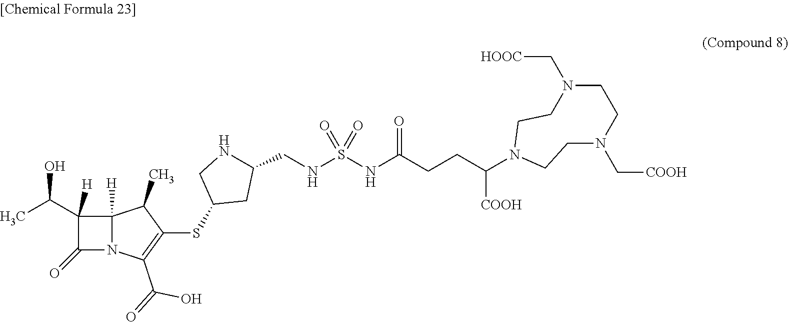 [SMILES: C[C@@H](O)[C@H]1C(=O)N2C(C(=O)O)=C(S[C@@H]3CN[C@H](CNS(=O)(=O)NC(=O)CCC(C(=O)O)N4CCN(CC(=O)O)CCN(CC(=O)O)CC4)C3)[C@H](C)[C@H]12]

Doripenem (Tokyo Chemical Industry, Ltd.) was dissolved in a 400 mM disodium hydrogen phosphate aqueous solution in such a manner that the concentration was 30 mM. Powdered NODA-GA-NHS (2,2′-(7-(1-carboxy-4-((2,5-dioxopyrrolidin-1-yl)oxy)-4-oxobutyl)-1,4,7-triazonane-1,4-diyl)diacetic acid (CheMatech)) was added to the solution in such a manner that the final concentration was 40 mM, thereby performing 30 minutes of reaction at 37° C. The reaction solution was purified by reverse-phase HPLC under the following conditions to obtain compound 7 yield: 75%).

Reverse phase column: YMC-Triat C18 Plus column (4.6×250 mm); column temperature: 35° C.; mobile phase (2 liquids): mobile phase A (0.1% formic acid aqueous solution), mobile phase B (acetonitrile); gradient: increase from 0.2% B to 40% B for 22 minutes, keep it at 40% B for 1 minute, and then return it to 0.2% B over 1 minute. Flow rate: 0.8 ml/min; detection: 254 nm; sample injection volume: 1 ml.

### Test Example 1

Based on the description of NON PATENT LITERATURE 5, a recombinant metallo-β-lactamase (recombinant IMP-1) was prepared. “NG_049172” of Database: RefSeq as the IMP-1 gene was incorporated into vector pET29a (Novagen), and then expressed in E. coli BL21(DE3) (Invitrogen).

Recombinant IMP-1 was dissolved in a 50 mM sodium phosphate buffer (pH 7.6) at a concentration of 0.25 μg/ml. Furthermore, zinc chloride was added to the solution in such a manner that the concentration was 0.1 μM. The resultant in this state was subjected to preincubation at 37° C. for 10 minutes. DTPA-cefalexin (compound 1) was added thereto in such a manner that concentrations were 1, 5, and 10 μM, and then 100 μM meropenem (Wako Pure Chemical Industries, Ltd.) was added. After 1 hour of incubation at 37° C. residual meropenem was quantified by tandem mass spectrometry. Meropenem was quantified by a multiple reaction monitoring method. The measurement conditions are as follows.

Reverse phase column: YMC-Triat C18 Plus column (2.1×50 mm); column temperature: 45° C.; mobile phase (2 liquids): mobile phase A (0.1% formic acid aqueous solution), mobile phase B (acetonitrile); gradient: increase from 1% B to 80% B for 10 minutes, keep it at 80% B for 0.5 minutes, and then return it to 1% B over 1 minute. Flow rate: 0.2 mil/min; sample injection volume: 10 μl. Detection: multiple reaction monitoring, parent ion at 384.2, child ion at 68.1; measured in a positive ion mode.

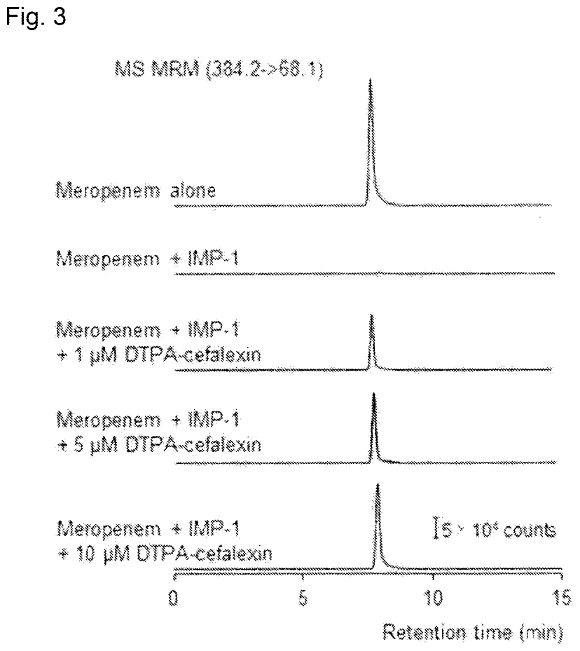

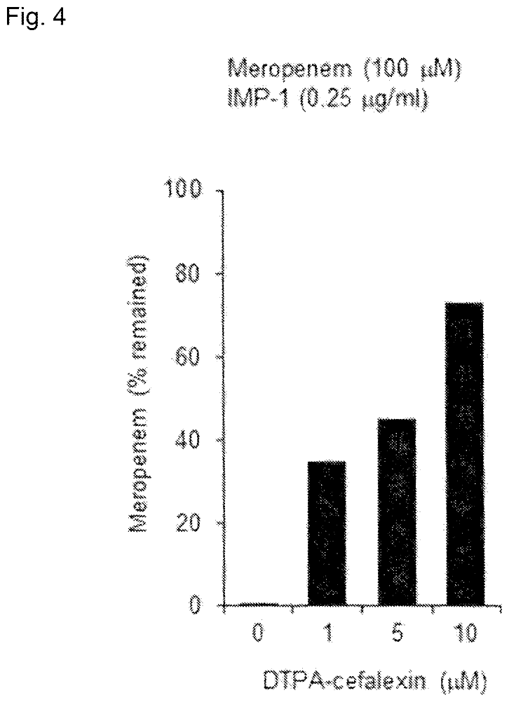

FIGS. 3 and 4 depict the results. A meropenem peak was detected at around 7.5 minutes in FIG. 3. Incubation with IMP-1 completely degraded meropenem. Addition of DTPA-cefalexin thereto inhibited the degradation of meropenem.

FIG. 4 depicts a graph showing the results of quantifying the peak area of meropenem detected in FIG. 3. The peak of meropenem alone is expressed as 100%.

### Test Example 2

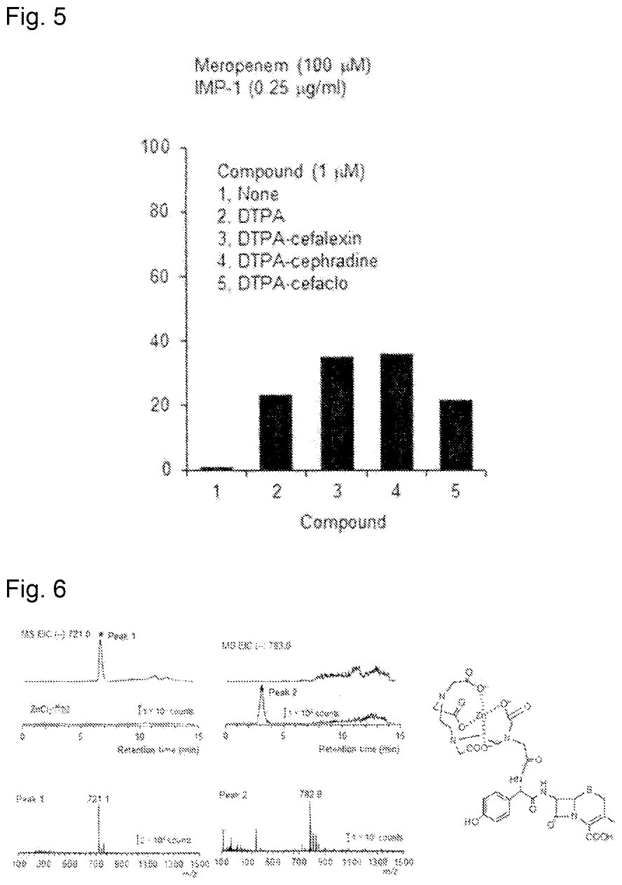

A test was conducted using DTPA, DTPA-cephradine, and DTPA-cefachlor under the same conditions as in Test Example 1, and the inhibitory effect on meropenem degradation by IMP-1 was evaluated when each sample was added at 1 μM. FIG. 5 depicts the results.

### Test Example 3

DTPA-cefalexin (100 μM) and zinc chloride (ZnCl₂, 100 μM) were reacted in a 50 mM sodium phosphate buffer (pH 7.6) at 37° C. for 1 hour. For comparison, DTPA-cefalexin alone was similarly incubated at 37° C. for 1 hour. After that, mass spectrometry confirmed the formation of a complex of DTPA-cefalexin and zinc. Measurement was performed in a negative mode. FIG. 6 depicts the results. DTPA-cefalexin was detected at m/z 721 (peak 1). On the other hand, when zinc was coordinated to DTPA-cefalexin, the resultant with the molecular weight of zinc added thereto was detected at m/z 783 (peak 2).

### Test Example 4

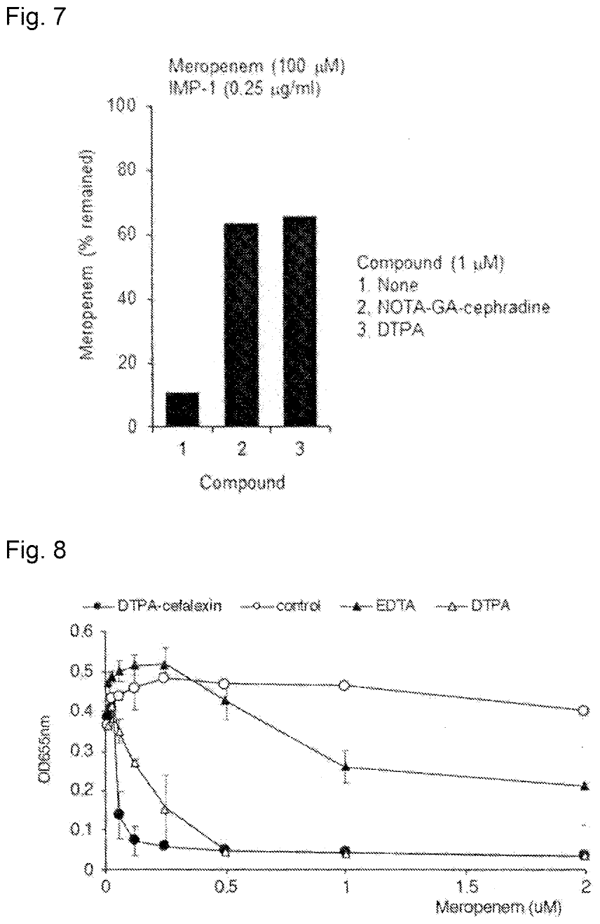

A test was conducted using DTPA and NOTA-GA-cephradine under the same conditions as in Test Example 1, and the inhibitory effect on the degradation of meropenem by IMP-1 was evaluated when each sample was added at 1 μM. FIG. 7 depicts the results.

### Test Example 5

Based on the description of NON PATENT LITERATURE 5, IMP-1-expressing E. coli was prepared.

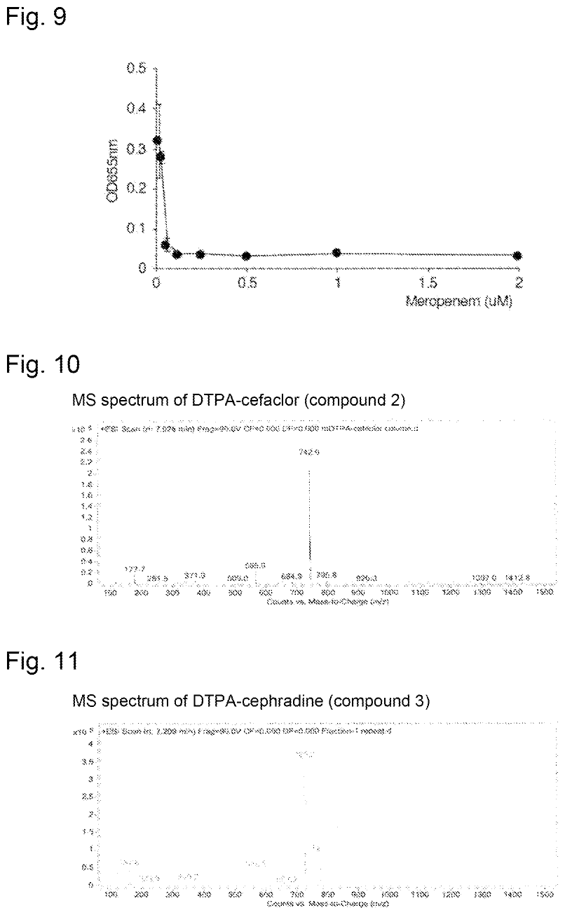

The effects of DTPA-cefalexin and NOTA-cefalexin on the susceptibility of IMP-1-expressing E. coli to a carbapenem antibacterial agent (meropenem) were evaluated by the following method. The IMP-1-expressing E. coli was cultured overnight with shaking in LB medium containing 100 μg/ml ampicillin. The bacterial solution was diluted to 1/200 with LB medium, used as a test bacterial solution, and then seeded in a 96-well plate. Meropenem (Wako Pure Chemical Industries, Ltd.) was added to the bacterial solution, thereby performing two-step dilution from a maximum of 2 μM. The bacteria were cultured in an incubator at 37° C. for one day, and the growth at that time was measured by turbidity (655 nm). FIGS. 8 and 9 depict the results.

FIG. 8 depicts the susceptibility to meropenem when DTPA-cefalexin was added at 25 μM, or EDTA or DTPA was added at 25 μM. FIG. 9 depicts susceptibility to meropenem when NOTA-cefalexin was added at 25 μM. Those denoted as “control” in the figure show the results of adding only meropenem. It was confirmed that when DTPA-cefalexin and NOTA-cefalexin coexist, metallo-β-lactamase-expressing bacteria are killed by the effect of the carbapenem antibacterial agent.

### Test Example 6

DTPA-conjugated cefalexin (compound 1)'s effect of enhancing the drug susceptibility of pathogenic bacteria was examined by an in vivo experiment using bacteria-infected mice. To prepare a compromised Leukopenia mouse model, 0.1 mL of cyclophosphamide monohydrate (Sigma-Aldrich) prepared with PBS was administered intraperitoneally to 4-week-old male ddY mice (Japan SLC) at 250 mg/kg at 4 days before infection. IMP-1-expressing Klebsiella pneumoniae used herein was a clinical isolate provided by Kumamoto University Hospital. The bacteria were cultured overnight with shaking in LB medium at 37° C., then diluted 50-fold with LB medium, and further cultured with shaking, so that the turbidity (600 nm) was 1.5 or higher. The bacteria were centrifuged to remove the medium, washed twice with PBS, and then diluted with PBS to 5×10⁵ CFU/mL. In the mouse infection experiment, each Leukopenia mouse was infected with 0.1 mL (5×10⁴ CFU) of IMP-1-expressing Klebsiella pneumoniae by intraperitoneal injection. Thirty minutes after infection, 0.1 mL of a mixed solution containing meropenem (Wako Pure Chemical Industries, Ltd.) and compound 1 (DTPA-CEF) was subcutaneously administered to each mouse of a treatment mouse group at 10 mg/kg and 50 mg/kg, respectively. As a control group, 0.1 mL of meropenem (10 mg/kg) or PBS as a solvent was subcutaneously administered. Forty-eight hours after infection, mice were assessed for viability. FIG. 17 depicts the results. The number of mice of each group is as follows: a PBS administration group (7 mice) to which PBS was administered, a meropenem administration group (4 mice) to which meropenem was administered, and a meropenem and DTPA-CEF combined administration group (4 mice) to which meropenem and DTPA-CEF were administered in combination.

IMP-1-expressing Klebsiella pneumoniae exhibited lethality to Leukopenia model mice, and the percent survival after 48 hours decreased to 14% (FIG. 17, PBS administration group). All mice of the meropenem administration group died, but the percent survival was improved to 50% in the treatment group administered with meropenem and compound 1 (FIG. 17, MEPM administration group and MEPM+DTPA-CEF administration group).

### Test Example 7

The effect of NODA-GA-conjugated doripenem (compound 8) on the susceptibility of a Pseudomonas aeruginosa clinical isolate to a carbapenem antibacterial agent was evaluated by the following method. The Pseudomonas aeruginosa clinical isolate was cultured overnight with shaking in LB medium. The bacterial solution was diluted to 1/1000 with LB medium, used as a test bacterial solution, and then seeded in a 96-well plate. Meropenem (Wako Pure Chemical Industries, Ltd.) was added to this bacterial solution in such a manner that the concentration was 2 μg/ml. The effect of NODA-GA-doripenem alone was also examined. The bacteria were cultured in an incubator at 37° C. for one day, and the growth at that time was measured by turbidity (655 nm). Results are shown in the table below.

TABLE 1
Antibacterial effect of compound 8 (NODA-GA-doripenem)
and combined effect of meropenem (MEPM) and compound
8 on multidrug-resistant Pseudomonas aeruginosa

| | | NODA-GA- | |
|---|---|---|---|
| Strain | MEPM | doripenem | Combination |
| NM1 | X | Δ | ◯ |
| NM2 | X | Δ | Δ |
| NM3 | X | ◯ | ◯ |
| NM4 | X | ◯ | ◯ |
| NM5 | X | X | X |
| MR1 | X | ◯ | ◯ |
| MR2 | X | ◯ | ◯ |
| MR3 | X | Δ | ◯ |
| MR4 | X | Δ | ◯ |
| MR5 | X | ◯ | ◯ |
| MR6 | X | Δ | ◯ |
| MR7 | X | ◯ | ◯ |
| MR8 | X | X | X |
| MR9 | X | ◯ | ◯ |
| MR10 | X | Δ | ◯ |
| MR11 | X | X | X |
| MR12 | X | X | X |
| MR13 | X | X | X |
| MR14 | X | Δ | Δ |
| MR15 | X | ◯ | ◯ |
| MR16 | X | X | X |
| 808#1 | X | X | X |
| 808#2 | X | X | ◯ |

In Table 1, ◯ indicates that bacterial growth was suppressed in all 3 wells. Δ indicates that bacterial growth was suppressed in 2 wells out of 3 wells. x indicates that the number of wells in which suppression of bacterial growth was confirmed was 1 or less. MEPM in Table 1 is meropenem (2 μg/ml) administered alone; NODA-GA-doripenem is compound 8 (20 μM; 15.5 μg/ml) administered alone; Combination means a combined use of meropenem (2 μg/ml) and compound 8 (20 μM; 15.5 μg/ml).

### Test Example 8

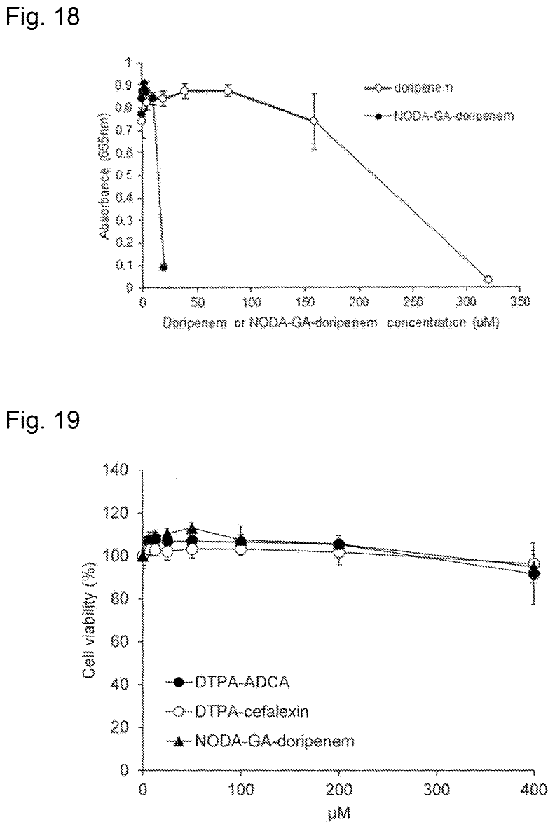

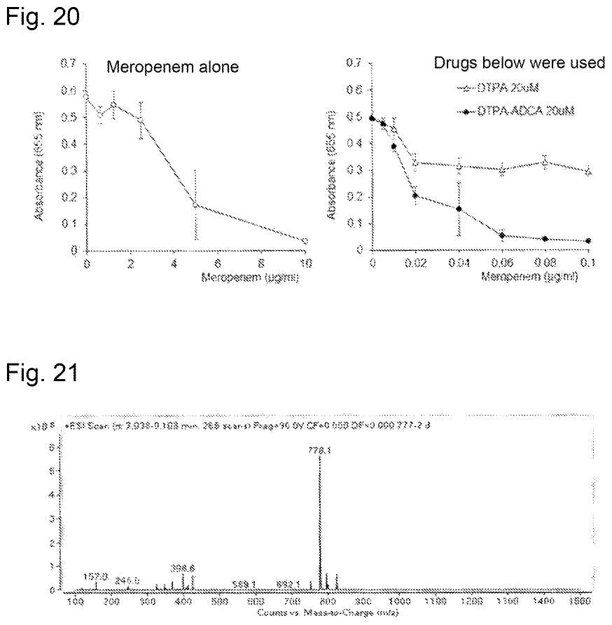

Multidrug-resistant Pseudomonas aeruginosa strain MR4 was cultured with doripenem or compound 8, and the bacterial load was assessed by turbidity after 24 hours. FIG. 18 depicts the results. It was confirmed that the anti-Pseudomonas aeruginosa effect of NODA-GA-conjugated doripenem was significantly improved compared to that of doripenem.

### Test Example 9

Cytotoxicity of compound 6 (DTPA-conjugated ADCA), the same of compound 1 (DTPA-conjugated cefalexin), and the same of compound 8 (NODA-GA-conjugated doripenem) were evaluated using a 3-(4,5-dimethylthia-2-yl)-2,5-tetrazolium bromide (MTT) method. HeLa cells were dispensed into 96-well plates at 1×10⁴ cells per well, and cultured overnight at 37° C. under 5% carbon dioxide gas circulation. Test compounds were each added to the cells, thereby performing two-step dilution from a maximum of 400 μM. After 6 hours of treatment with the compounds, medium exchange was performed, MIT dissolved in PBS in such a manner that the concentration had been 7.5 mg/mL was added, and the reaction was further performed for 2 hours. Thereafter, the supernatant was removed from each well, and an isopropanol hydrochloride solution was added to dissolve formazan crystals. Cytotoxicity was calculated from the difference between the absorbance increase at 490 nm due to formazan elution and the absorbance at 655 nm. FIG. 19 depicts the results. The results were expressed as 100% when no compound had been added.

### Test Example 10

The enhancing effect of DTPA-ADCA on the susceptibility of E. coli clinical isolate expressing IMP-1 to a carbapenem antibacterial agent (meropenem) was evaluated by the following method. The E. coli clinical isolate was cultured overnight with shaking in LB medium. The bacterial solution was diluted to 1/1000 with LB medium, used as a test bacterial solution, and then seeded in a 96-well plate. Meropenem (Wako Pure Chemical Industries, Ltd.) was added to the bacterial solution, thereby performing two-step dilution from a maximum of 10 μg/ml. The bacteria were cultured in an incubator at 37° C. for one day, and growth at that time was measured by turbidity (655 nm). The meropenem concentration at which no bacterial growth had been observed was defined as the minimum inhibitory concentration.

The effect of DTPA-ADCA on the susceptibility of an E. coli clinical isolate to meropenem was evaluated by the following method. The E. coli clinical isolate was cultured overnight with shaking in LB medium. The bacterial solution was diluted to 1/1000 with LB medium, used as the test bacterial solution, and then seeded in a 96-well plate. Meropenem (Wako Pure Chemical Industries, Ltd.) was added to this bacterial solution in the range of 0.1 μg/ml to 0.005 μg/ml. Here, DTPA or DTPA-ADCA was used in combination therewith at a concentration of 20 mM, and the effect on bacterial growth was examined. The bacteria were cultured in an incubator at 37° C. for one day, and the growth at that time was measured by turbidity (655 nm).

FIG. 20 depicts the results. Multidrug-resistant E. coli was cultured with compounds having the indicated concentrations and the bacterial load was assessed by turbidity after 24 hours. Meropenem alone resulted in a minimum inhibitory concentration of 10 μg/ml (left graph). Combined use of DTPA-ADCA (20 μM) reduced the minimum inhibitory concentration of meropenem to 10 μg/ml and enhanced the antibacterial activity by 100 times. DTPA by itself had no such effect (right graph).

1. A compound represented by formula (I), formula (II), or formula (IV): 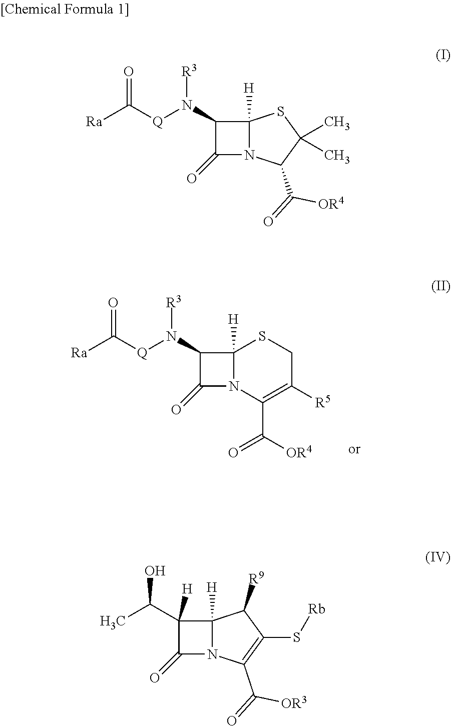
or a pharmaceutically acceptable salt thereof, wherein
Q is a direct bond or a group: —N(—R²)—CH(—R¹)—C(═O)—, wherein a nitrogen atom of the group is linked to a carbonyl group described in formula (I) or formula (II);
R¹ is phenyl optionally substituted with one or more substituents selected from X¹, 5- or 6-membered heteroaryl optionally substituted with one or more substituents selected from X¹, C₁₋₁₀ alkyl optionally substituted with one or more substituents selected from X², C₂₋₁₀ alkenyl optionally substituted with one or more substituents selected from X², C₂₋₁₀ alkynyl optionally substituted with one or more substituents selected from X², C₃₋₁₀ cycloalkyl optionally substituted with one or more substituents selected from X², or C₆₋₁₀ cycloalkanedienyl optionally substituted with one or more substituents selected from X³;
R² is a hydrogen atom or C₁₋₆ alkyl;
R³ is a hydrogen atom or C₁₋₆ alkyl;
R⁴ is a hydrogen atom, C₁₋₆ alkyl optionally substituted with one or more substituents selected from X⁴, or 5- or 6-membered non-aromatic heterocyclyloxy optionally substituted with one or more substituents selected from X⁵, wherein the heterocyclyl of the non-aromatic heterocyclyloxy is optionally fused with a benzene ring;
R⁵ is a hydrogen atom, a halogen atom, C₁₋₆ alkyl, C₁₋₆ alkoxy, C₂₋₆ alkenyl optionally substituted with R⁶, or methyl substituted with R⁷;
R⁶ is phenyl optionally substituted with one or more substituents selected from X¹, or 5- or 6-membered heteroaryl optionally substituted with one or more substituents selected from X¹;
R⁷ is 5- or 6-membered heteroarylsulfanyl optionally substituted with one or more substituents selected from X⁵, 5 or 6-membered heteroaryloxy optionally substituted with one or more substituents selected from X⁵, phenylsulfanyl optionally substituted with one or more substituents selected from X⁵, phenyloxy optionally substituted with one or more substituents selected from X⁵, (C₁₋₆ alkyl)carbonyloxy, carbamoyloxy, pyridinium-1-yl optionally fused with C₅₋₇ cycloalkyl and optionally substituted with X⁵, or 1-methylpyrrolidinium-1-yl optionally substituted with X⁵;
X¹ is amino, hydroxy, C₁₋₆ alkyl, C₁₋₆ alkoxy, a halogen atom, or phenyl optionally substituted with one or more halogen atoms or hydroxy;
X² is amino, hydroxy, C₁₋₆ alkoxy, a halogen atom, carboxy, or (C₁₋₆ alkoxy)carbonyl;
X³ is C₁₋₆ alkyl or a halogen atom;
X⁴ is (C₁₋₆ alkyl)carbonyloxy or (C₁₋₆ alkoxy)carbonyloxy;
X⁵ is C₁₋₆ alkyl optionally substituted with R⁸, or carbamoyl;
X⁶ is C₁₋₆ alkyl, a halogen atom, (C₁₋₆ alkoxy)carbonyl, or carboxy;
R⁸ is hydroxy, sulfo, or carboxy;
R⁹ is a hydrogen atom or methyl;
Ra is a polyamine group represented by any of the following formulae: 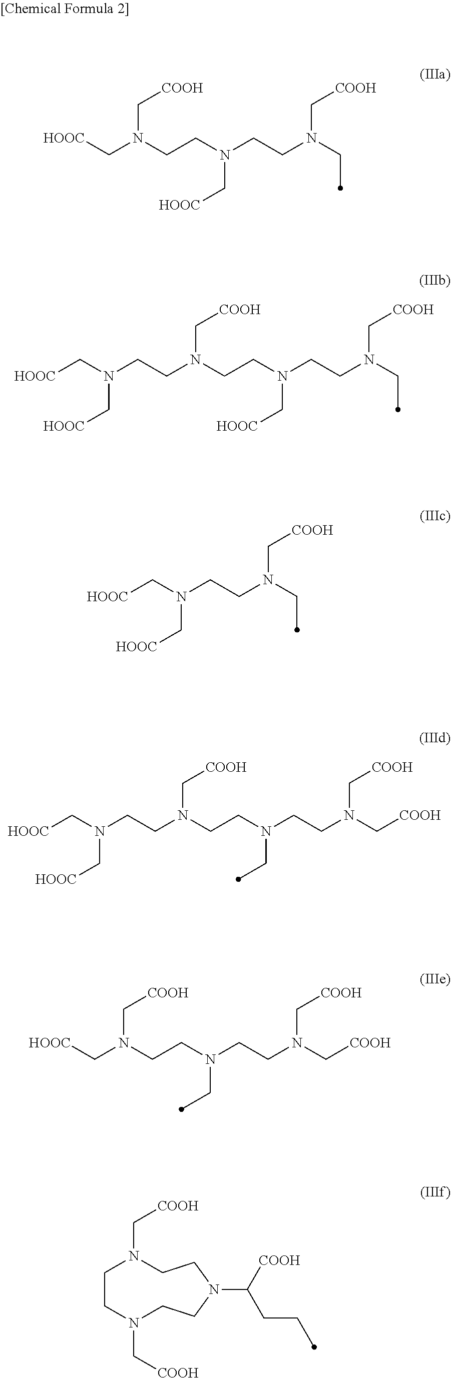 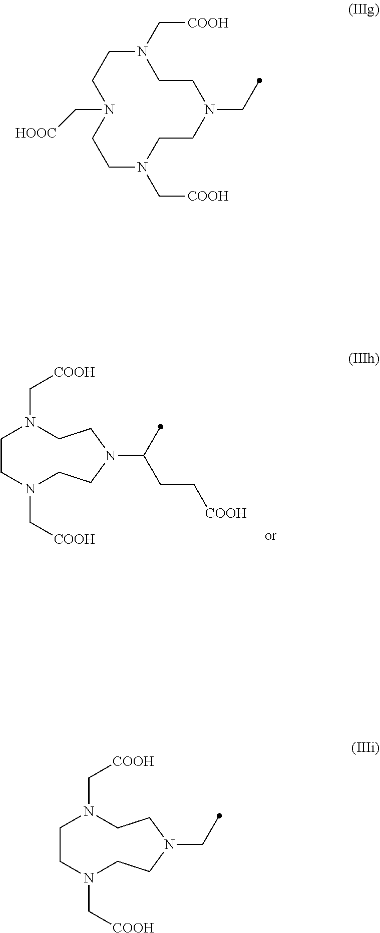
and ● indicates the bonding position; and
Rb is selected from a group represented by the following formula: 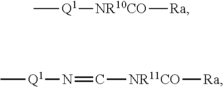 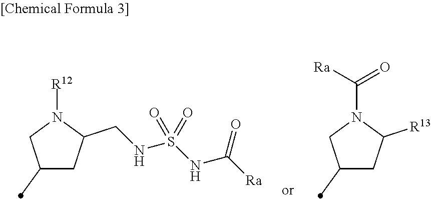
Q¹ is C₂₋₆ alkylene;
R¹⁰ is a hydrogen atom, C₁₋₆ alkyl, or —CH═NH;
R¹¹ and R¹² are each independently a hydrogen atom or C₁₋₆ alkyl;
R¹³ is a hydrogen atom, C₁₋₆ alkyl, —CH₂NHSO₂NH₂, or —CONR¹⁴R¹⁵, wherein the alkyl is optionally substituted with one or more substituents selected from —NR¹⁴R¹⁵ and hydroxy;
R¹⁴ is a hydrogen atom, C₁₋₆ alkyl, or phenyl optionally substituted with one or more substituents selected from X⁶; and
R¹⁵ is a hydrogen atom or C₁₋₆ alkyl.

2. The compound or the pharmaceutically acceptable salt thereof according to claim 1, wherein R¹ is phenyl optionally substituted with one or more substituents selected from X¹, 5- or 6-membered heteroaryl optionally substituted with one or more substituents selected from X¹, or C₆₋₁₀ cycloalkanedienyl optionally substituted with one or more substituents selected from X³.

3. The compound or the pharmaceutically acceptable salt thereof according to claim 1, wherein R⁴ is a hydrogen atom or C₁₋₆ alkyl substituted with one substituent selected from X⁴.

4. The compound or the pharmaceutically acceptable salt thereof according to claim 1, wherein Ra is a polyamine group represented by formula IIIa, formula IIIh or formula IIIi.

5. The compound or the pharmaceutically acceptable salt thereof according to claim 1, wherein R² is a hydrogen atom.

6. The compound or the pharmaceutically acceptable salt thereof according to claim 1, wherein the compound is represented by formula II.

7. The compound or the pharmaceutically acceptable salt thereof according to claim 1, wherein R⁵ is a hydrogen atom, a halogen atom, C₁₋₆ alkyl, C₁₋₆ alkoxy, or C₂₋₆ alkenyl.

8. The compound or the pharmaceutically acceptable salt thereof according to claim 1, wherein Rb is selected from a group represented by the following formula:

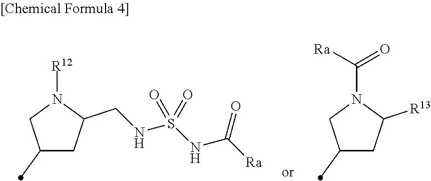

9. The compound or the pharmaceutically acceptable salt thereof according to claim 1, wherein Rb is selected from a group represented by the following formula:

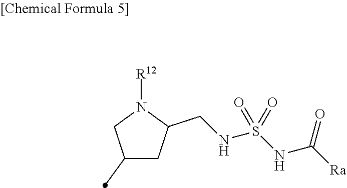

10. A pharmaceutical composition comprising the compound or the pharmaceutically acceptable salt thereof according to claim 1.

11. The pharmaceutical composition according to claim 10, for use in treating an infectious disease caused by a β-lactam antibiotic-resistant bacterium.

12. The pharmaceutical composition according to claim 10, wherein the resistant bacterium is a metallo-β-lactamase-expressing bacterium.

13. The pharmaceutical composition according to claim 10, for use in combination with a β-lactam antibiotic.

14. A metallo-β-lactamase inhibitor, comprising the compound or the pharmaceutically acceptable salt thereof according to claim 1.
